(19) United States
(12) Patent Application Publication
(10) Pub. No.: US 2022/0036481 A1
(43) Pub. Date: Feb. 3, 2022

(54) SYSTEM AND METHOD TO INTEGRATE EMOTION DATA INTO SOCIAL NETWORK PLATFORM AND SHARE THE EMOTION DATA OVER SOCIAL NETWORK PLATFORM

(71) Applicant: Steve CURTIS, Vancouver (CA)

(72) Inventor: Steve CURTIS, Vancouver (CA)

(21) Appl. No.: 17/278,516

(22) PCT Filed: Sep. 21, 2019
(86) PCT No.: PCT/IB2019/058002

§ 371 Date: Mar. 22, 2021

Related U.S. Application Data
(60) Provisional application No. 62/734,571, filed on Sep. 21, 2018.
(60) Provisional application No. 62/734,587, filed on Sep. 21, 2018.
(60) Provisional application No. 62/734,608, filed on Sep. 21, 2018.

(51) Int. Cl. G06Q 50/00; G16H 40/67; G16H 50/30; G16H 15/00; G06F 16/26; A61B 5/00; A61B 5/16; G06F 3/14; H04B 1/3827; H04L 12/58

(52) U.S. Cl. CPC G06Q 50/01; G16H 40/67; G16H 50/30; G16H 15/00; G06F 16/26; A61B 5/4857; G16H 50/20; A61B 5/165; A61B 5/0022; G06F 3/14; H04B 1/385; H04L 51/32; A61B 5/6802

(57) ABSTRACT

Disclosed is a system and method for integrating emotion data into a social network platform and share the emotion data over the social network platform connected through a communication network. The method includes the step of collecting biorhythm data of the user through a wearable user device. The method includes the step of receiving the biorhythm data of the users through a computing device communicatively connected with the wearable user device over the communication network. The method includes the step of integrating the emotion data through an integration module. The method includes the step of determining an emotional state of the user through an emotional state determination module. The method includes the step of analyzing and displaying emotional data of the users in real-time through an emotional data displaying module.

### TECHNICAL FIELD

The present invention relates to system and method to integrate emotion data into a social network platform to enhance communication among a plurality of users, and increase human connection and awareness between the users, in particular to a system and method to system and method to integrate emotion data into a social network platform and share the emotion data over the social network platform.

### BACKGROUND ART

With the advent of various social network platforms, people spend a tremendous amount of time on the internet to virtually interact with the other connected users. This specification recognizes that the mental states of the user can be evaluated to understand the reaction of the users towards the various activities happening around them. Mental states include a broad gamut from happiness to sadness, from contentedness to worry, from excitement to calmness, etc. These mental states are experienced in response to everyday events such as frustration during a traffic jam, boredom while standing in line, impatience while waiting for a cup of coffee, and even as people interact with their computers and the internet. Individuals may become rather perceptive and empathetic based on evaluating and understanding others' mental states but automated evaluation of mental states is far more challenging. An empathetic person may perceive another's being anxious or joyful and respond accordingly. The ability and means by which one person perceives another's emotional state may be quite difficult to summarize and has often been communicated as having a “gut feel.”

Many mental states, such as confusion, concentration, and worry, maybe identified to aid in the understanding of an individual or group of people. People can collectively respond with fear or anxiety, such as after witnessing a catastrophe. Likewise, people can collectively respond with happy enthusiasm, such as when their sports team obtains a victory. Certain facial expressions and head gestures may be used to identify a mental state that a person is experiencing. Limited automation has been performed in the evaluation of mental states based on facial expressions. Certain physiological conditions may provide telling indications of a person's state of mind and have been used in a crude fashion as in polygraph tests.

Likewise, people now have the ability to provide instant and continuous feedback in response to various social media such as pictures, websites, and the like. Such feedback can be provided on computers, tablets, smartphones, and other devices that access the internet. For example, “like” is a way to give positive feedback or to connect with things a person is interested in on the popular social media site Facebook®. In particular, the “like” button on Facebook® is a button a user may click on after looking at most content on Facebook®, which is then reported in newsfeeds to “friends”. Websites unrelated to Facebook® may also use a “like” button that enables a website visitor to click on the button to let his/her friends know that they like the site. For example, after clicking on the website's “like” button, a pop-up will request a login to Facebook® (or sign-up if not already a member) and a post on the user's Facebook® page will let his/her friends know that he/she likes the site. When used on a mobile device, such as a smartphone, the “like” button is merely an integrated hardware “Facebook®” button on the phone that does nothing more than taking the user to Facebook® when the button is pressed.

Similarly, the “Pin It” button on a computer or mobile device allows users to grab images and videos from around the web and add them to on-line pinboards created by the users. Other users can view the pinboards, comment, and “re-pin”. Capabilities have also been introduced to allow people to use mobile devices to interact with their environment. For example, location-based social networking websites allow users to “check-in” at venues using a mobile website, text messaging, or a device-specific application by selecting from a list of venues the application locates nearby. The location is based on GPS hardware in the mobile device or the network location provided by the application. Each check-in awards the user points or other types of rewards.

Even with these advances in technology, the ability to measure and evaluate the user experience, effectiveness, and the usability of social media, locations, or experiences has been limited. In fact, current methodologies for measuring or evaluating user experience, effectiveness, and usability of websites and other interactive internet and software media has thus far been limited to traditional self-report, i.e., relying on the user to use the “like” button and to accurately reflect his/her actual response to the social media, which may be subject to error, bias, or low compliance.

The popularity and growth of social network sites and services have increased dramatically over the last few years. Present social network sites include Facebook®, Google+®, Twitter®, MySpace®, YouTube®, LinkedIn®, Flicker®, Jaiku®, MYUBO®, Bebo® and the like. Such social networking (SNET) sites are typically web-based and organized around user profiles and/or collections of content accessible by members of the network. Membership in such social networks is comprised of individuals, or groupings of individuals, who are generally represented by profile pages and permitted to interact as determined by the social networking service.

In many popular social networks, especially profile-focused social networks, activity centers on web pages or social spaces that enable members to view profiles, communicate and share activities, interests, opinions, status updates, audio/video content, etc., across networks of contacts. Social networking services might also allow members to track certain activities of other members of the social network, collaborate, locate and connect with existing friends, former acquaintances, and colleagues, and establish new connections with other members.

Thus, a need in the art exists for a system and method that may integrate various sensors into computing devices to collaborate with the social network platforms to perform various functions such as eliminating the “like” button and replacing it with a continuous stream of emotional responses across all the experiences. A need also exists in the art for a biometrically enabled suite of applications that are built into smartphones, tablets, and other social media-enabled devices to determine when a user unconsciously likes (or dislikes) their current experience, e.g., a web page, “app”, song, video, location, or other experience, and also to remotely monitor the user's stress levels and well-being in real-time and specially based on the information of emotions of other users. Further, there is a need to provide a system and a method for a dynamic approach of tracking biorhythms in response to seeing other users' emotional data attached or represented in the posts and content shared on the social network platform or privately between two users on messaging/audio-recording application.

Thus, in view of the above, there is a long-felt need in the industry to address the aforementioned deficiencies and inadequacies.

Further limitations and disadvantages of conventional and traditional approaches will become apparent to one of skill in the art through comparison of described systems with some aspects of the present disclosure, as set forth in the remainder of the present application and with reference to the drawings.

### SUMMARY OF INVENTION

A system to integrate emotion data into a social network platform and share the emotion data over the social network platform connected through a communication network is provided substantially, as shown in and/or described in connection with at least one of the figures, as set forth more completely in the claims.

The present invention provides a method for integrating emotion data into a social network platform and shares the emotion data over the social network platform connected through a communication network. The method includes the step of collecting biorhythm data of the user through a wearable user device configured to be worn on the user's body, near the body, or placed in the user's body (implantable). The method includes the step of receiving the biorhythm data of the users through a computing device communicatively connected with the wearable user device over the communication network. The method includes the step of integrating the emotion data through an integration module. The method includes the step of determining an emotional state of the user through an emotional state determination module. The method includes the step of analyzing and displaying emotional data of the users in real-time through an emotional data displaying module.

The integration module performs a plurality of steps that initiates with a step of collecting physiological data of at least one physiological property of the user through a physiological data collection engine. The method includes the step of processing the physiological data into at least one biosignal through a biosignal generating engine. The method includes the step of monitoring and measures the biosignal for determining at least one score pertaining to at least one of the emotion of the user, and stress of the user through a score calculating engine. The method includes the step of integrating the scores with at least one of a social media post associated with the social network platform, a textual conversation, and a multimedia conversation (audio, video) and overlays information pertaining to emotion of the user, and stress of the user through a social integrating and information overlay engine.

The emotional state determination module performs a plurality of steps that initiates with a step of analyzing the emotional state of the user on receiving the biorhythm data from the wearable user device through an analysis module. The method includes the step of associating the analyzed emotional state of the user corresponding to at least one of one or more posts being shared by the user, one or more pieces of content being shared to the user, one or more reactions to the posts, and one or more responses to the posts over the social network platform through an emotive module. The method includes the step of displaying a representation of the emotional state of the user in the social network platform through a display module.

The emotional data displaying module performs a plurality of steps that initiates with a step of analyzing the biorhythm data and computing an emotional score of the user to generate one or more insights through an algorithmic module. The emotional score is indicative of the emotional state of the user during the interactions. The method includes the step of visually representing a plurality of emotional cycles for a specific time duration for the user through a visualization module. The visualization module displays the insights and emotional scores of the users on the computing device associated with the users.

In an aspect, the emotive module facilitates the user to initiate a command to associate the emotional state of the user corresponding to the posts being shared by the user, and the content being shared to the user.

In an aspect, the display module displays the representation of the emotional state with respect to the post being shared by the user, and the content shared to the user on receiving a request command from the user.

In an aspect, the visualization module displays emotional data in a plurality of manners on at least one of a two dimensional (2D) graph, and a three dimensional (3D) graphs by using at least one of a plurality of alpha-numeric characters, a plurality of geometric shapes, a plurality of holograms, and a plurality of symbols which include colors or moving shapes.

In an aspect, the emotion data can be obtained by using one or more bio-signal sensors, such as electroencephalogram (EEG) sensors, galvanometer sensors, electrocardiograph sensors, heart rate sensors, eye-tracking sensors, blood pressure sensors, pedometers, gyroscopes, and any other type of sensor. The sensors may be connected to the wearable user device, such as a wearable headset, ring, watch, bracelet and/or headband worn by the user. The sensors may be connected to the wearable user device by wires or wirelessly.

In an aspect, the medical professionals may see an overlay of the user's (patient) emotional state/biometric information over a visual diary of their day. This information could be used in understanding patients, recognizing patterns, and visualizing situations. Similarly, the overlay of the user's emotional state/conscious level can be illustrated on any web or mobile application by integrating the biorhythm data collected from the user in real-time.

In an aspect, the information regarding the second user's emotions can be published and/or posted on any social media websites, portal or channel. The information can be overlaid and/or integrated with text messages, Skype chats or calls, and/or any other form of instant messaging or communication.

In an aspect, the users emotions can be dynamically tracked based on the emotions of the secondary users.

In an aspect, the score may be represented as a numeric value, and as a picture illustrating emotions like anger, sad, and happy, etc.

Another aspect of the present invention relates to a system to integrate emotion data into a social network platform and share the emotion data over the social network platform connected through a communication network. The system includes a wearable user device and a computing unit. The wearable user device configured to be worn on the user's body, near the body or placed in the user's body (implantable) to collect biorhythm data of the user. The computing unit is communicatively connected with the wearable user device to receive the biorhythm data of the users over a communication network. The computing unit includes a processor, and a memory communicatively coupled to the processor. The memory includes an integration module, an emotional state determination module, and an emotional data displaying module. The integration module integrates the emotion data into the social network platform. The emotional state determination module determines an emotional state of the user. The emotional data displaying module analyzes and display emotional data of the users in real-time.

The integration module includes a physiological data collection engine, a biosignal generating engine, a score calculating engine and a social integrating and information overlay engine. The physiological data collection engine collects physiological data of at least one physiological property of the user. The biosignal generating engine processes the physiological data into at least one biosignal. The score calculating engine monitors and measures the biosignal for determining at least one score pertaining to at least one of the emotion of the user, and stress of the user. The social integrating and information overlay engine integrates the scores with at least one of a social media post associated with the social network platform, a textual conversation, and a multimedia conversation (audio, video) and overlays information pertaining to the emotion of the user, and stress of the user.

The emotional state determination module includes an analysis module, an emotive module, and a display module. The analysis module analyses the emotional state of the user on receiving the biorhythm data from the wearable user device. The emotive module associates the analyzed emotional state of the user corresponding to at least one of one or more posts being shared by the user, one or more pieces of content being shared to the user, one or more reactions to the posts, and one or more responses to the posts over the social network platform. The display module displays a representation of the emotional state of the user in the social network platform.

The emotional data displaying module includes an algorithmic module and a visualization module. The algorithmic module analyzes the biorhythm data and computes an emotional score of the user to generate one or more insights. The emotional score is indicative of the emotional state of the user during the interactions. The visualization module visually represents a plurality of emotional cycles for a specific time duration for the user. The visualization module displays the insights and emotional scores of the users on the computing device associated with the users.

Accordingly, one advantage of the present invention is that it enhances communication among a plurality of users, and increase human connection and awareness between the users.

Accordingly, one advantage of the present invention is that it monitors the emotions of persons using biorhythms and sharing such information with others over the internet. More specifically, the present invention relates to measuring the user's biorhythm and sharing on the social media network.

Accordingly, one advantage of the present invention is that it provides a social platform to the users where they share their emotional data and allow other users to visualize the same to improve and work on their emotional state.

Accordingly, one advantage of the present invention is that it determines the user's emotional state based on the user's biorhythm and relaying the emotional state to the other users, via a social network platform.

Accordingly, one advantage of the present invention is that it provides the emotional data of the users periodically to help the users to optimize their emotional and psychological state over time and allowing them to feel more consistently in a positive state.

Other features of embodiments of the present invention will be apparent from the accompanying drawings and from the detailed description that follows.

Yet other objects and advantages of the present invention will become readily apparent to those skilled in the art following the detailed description, wherein the preferred embodiments of the invention are shown and described, simply by way of illustration of the best mode contemplated herein for carrying out the invention. As we realized, the invention is capable of other and different embodiments, and its several details are capable of modifications in various obvious respects, all without departing from the invention. Accordingly, the drawings and description thereof are to be regarded as illustrative in nature, and not as restrictive.

### BRIEF DESCRIPTION OF DRAWINGS

In the figures, similar components and/or features may have the same reference label. Further, various components of the same type may be distinguished by following the reference label with a second label that distinguishes among the similar components. If only the first reference label is used in the specification, the description applies to any one of the similar components having the same first reference label irrespective of the second reference label.

FIG. 1 illustrates a block diagram of the present system to integrate emotion data into a social network platform and share the emotion data over the social network platform connected through a communication network, in accordance with one embodiment of the present invention.

FIG. 2 illustrates a network implementation of the present system, in accordance with one embodiment of the present invention.

FIG. 3 illustrates a block diagram of the various modules within a memory of a computing device, in accordance with another embodiment of the present invention.

FIG. 4 illustrates a flowchart of the method for integrating emotion data into a social network platform and share the emotion data over the social network platform connected through a communication network, in accordance with an alternative embodiment of the present invention.

FIG. 5 illustrates a flowchart of the plurality of steps performed by an integration module, in accordance with an alternative embodiment of the present invention.

FIG. 6 illustrates a flowchart of the plurality of steps performed by an emotional state determination module, in accordance with an alternative embodiment of the present invention.

FIG. 7 illustrates a flowchart of the plurality of steps performed by an emotional data displaying module, in accordance with an alternative embodiment of the present invention.

### DESCRIPTION OF EMBODIMENTS

The present disclosure is best understood with reference to the detailed figures and description set forth herein. Various embodiments have been discussed with reference to the figures. However, those skilled in the art will readily appreciate that the detailed descriptions provided herein with respect to the figures are merely for explanatory purposes, as the methods and systems may extend beyond the described embodiments. For instance, the teachings presented and the needs of a particular application may yield multiple alternative and suitable approaches to implement the functionality of any detail described herein. Therefore, any approach may extend beyond certain implementation choices in the following embodiments.

References to “one embodiment,” “at least one embodiment,” “an embodiment,” “one example,” “an example,” “for example,” and so on indicate that the embodiment(s) or example(s) may include a particular feature, structure, characteristic, property, element, or limitation but that not every embodiment or example necessarily includes that particular feature, structure, characteristic, property, element, or limitation. Further, repeated use of the phrase “in an embodiment” does not necessarily refer to the same embodiment.

Methods of the present invention may be implemented by performing or completing manually, automatically, or a combination thereof, selected steps or tasks. The term “method” refers to manners, means, techniques and procedures for accomplishing a given task including, but not limited to, those manners, means, techniques, and procedures either known to, or readily developed from known manners, means, techniques and procedures by practitioners of the art to which the invention belongs. The descriptions, examples, methods, and materials presented in the claims and the specification are not to be construed as limiting but rather as illustrative only. Those skilled in the art will envision many other possible variations within the scope of the technology described herein.

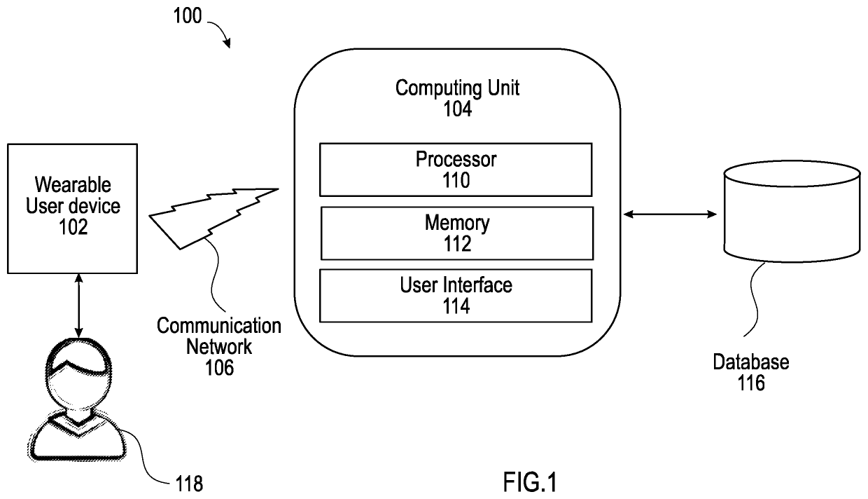

FIG. 1 illustrates a block diagram of the present system 100 to integrate emotion data into a social network platform and share the emotion data over the social network platform connected through a communication network, in accordance with one embodiment of the present invention. The system 100 includes a wearable user device 102, and a computing device 104. The wearable user device 102 is configured to be worn on the user's body, near the body, or placed in the user's body (implantable) to collect biorhythm data of the user 118. Examples of the wearable user device 102 include but not limited to the implantable, wireless sensor device, smartwatch, smart jewelry, fitness tracker, smart cloth, etc. In an embodiment, the wearable user device 102 includes various sensors to detect one or more parameters pertaining to the emotions of the user 118. In an embodiment, the wearable user device 102 may include a flexible body that can be secured around the user's 118 body to collect the biorhythm data. In an embodiment, the wearable user device 102 may include an accelerometer and a gyroscope to collect the biorhythm data. In an embodiment, and the wearable user device 102 may including a securing mechanism to secure the wearable user device 102 may in a closed loop around a wrist of the user 118. Further, the wearable user device 102 may be any wearable such as an on-body sticker or 3d-printed device that is directly printed on the skin, or a device that placed on the body with an adhesive. The wearable user device 102 may utilize various wired or wireless communication protocols to establish communication with the computing unit 104.

The computing device 104 is communicatively connected with the wearable user device 102 to receive the biorhythm data of the users over a communication network 106.

Communication network 106 may be a wired or a wireless network, and the examples may include but are not limited to the Internet, Wireless Local Area Network (WLAN), Wi-Fi, Long Term Evolution (LTE), Worldwide Interoperability for Microwave Access (WiMAX), General Packet Radio Service (GPRS), Bluetooth (BT) communication protocols, Transmission Control Protocol and Internet Protocol (TCP/IP), User Datagram Protocol (UDP), Hypertext Transfer Protocol (HTTP), File Transfer Protocol (FTP), ZigBee, EDGE, infrared (IR), Z-Wave, Thread, 5G, USB, serial, RS232, NFC, RFID, WAN, and/or IEEE 802.11, 802.16, 2G, 3G, 4G cellular communication protocols.

Examples of the computing device 104 include but not limited to a laptop, a desktop, a smartphone, a smart device, a smartwatch, a phablet, and a tablet. The computing device 104 includes a processor 110, a memory 112 communicatively coupled to the processor 110, and a user interface 114. The computing device 104 is communicatively coupled with a database 114. The database 116 receives and stores the emotional data and referral data which can be used for further analysis and prediction so that the present system can learn and improve the analysis by using the historical emotional data. Although the present subject matter is explained considering that the present system 100 is implemented on a cloud device, it may be understood that the present system 100 may also be implemented in a variety of computing systems, such as an Amazon elastic compute cloud (Amazon EC2), a network server, and the like.

Processor 110 may include at least one data processor for executing program components for executing user- or system-generated requests. A user may include a person, a person using a device such as those included in this invention, or such a device itself. Processor 110 may include specialized processing units such as integrated system (bus) controllers, memory management control units, floating point units, graphics processing units, digital signal processing units, etc.

Processor 110 may include a microprocessor, such as AMD® ATHLON® microprocessor, DURON® microprocessor OR OPTERON® microprocessor, ARM's application, embedded or secure processors, IBM® POWERPC®, INTEL'S CORE® processor, ITANIUM® processor, XEON® processor, CELERON® processor or other line of processors, etc. Processor 110 may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), etc.

Processor 110 may be disposed of in communication with one or more input/output (I/O) devices via an I/O interface. I/O interface may employ communication protocols/methods such as, without limitation, audio, analog, digital, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.n/b/g/n/x, Bluetooth, cellular (e.g., code-division multiple access (CDMA), high-speed packet access (HSPA+), global system for mobile communications (GSM), long-term evolution (LTE), WiMax, or the like), etc.

Memory 112, which may be a non-volatile memory or a volatile memory. Examples of non-volatile memory may include, but are not limited to flash memory, a Read Only Memory (ROM), a Programmable ROM (PROM), Erasable PROM (EPROM), and Electrically EPROM (EEPROM) memory. Examples of volatile memory may include but are not limited Dynamic Random Access Memory (DRAM), and Static Random-Access memory (SRAM).

The user interface 114 may present the integrated emotion data, and shared emotion data as per the request of an administrator of the present system. In an embodiment, the user interface (UI or GUI) 114 is a convenient interface for accessing the social network platform and viewing the biorhythm data of the connected users. The biorhythm data includes but is not limited to heart rate, heart rate variability, electrodermal activity (EDA)/Galvanic skin response (GSR), breathing rate, 3D accelerometer data, and gyroscope data, body temperature, among others. The biorhythm data can be processed to generate the signals based on mathematical description or algorithms. The algorithms may be introduced via software. There is potential that data is processed on the wearable user device end. Data may also be stored there temporarily before acted upon.

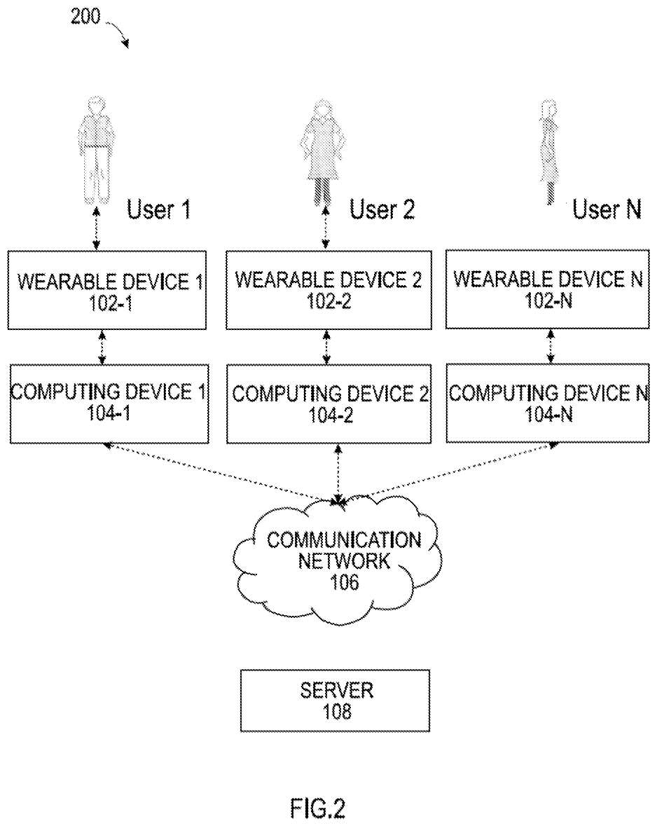

FIG. 2 illustrates a network implementation 200 of the present system, in accordance with one embodiment of the present invention. FIG. 2 is explained in conjunction with FIG. 1. The computing devices 104-1, 104-2, and 104-N are communicatively connected with the wearable user devices 102-1, 102-2, and 102-N to receive the biorhythm data of the users over the communication network 106. A server 108 stores and processes the integrated and shared emotion data. The computing device 104 or wearable user device 102 may initiate a sound notification (any type of sound). Based on the user's current emotional state score, different sounds should be issued by one or more of the wearable user devices 102 to inform the users to do one of several different behaviors. It may be appreciated that behavior may not be limited to one behavior, and sound could signal a plurality (multiple) of actions. The behavior associated with the sound should help the user change their behavior to move closer to the user's desired/preset emotional state, or move towards changing a more specific biorhythm.

In an aspect, the network architecture of the wearable user device 102 and the computing device 104 can include one or more Internet of Things (IoT) devices. In a typical network architecture of the present disclosure can include a plurality of network devices such as transmitter, receivers, and/or transceivers that may include one or more IoT devices.

In an aspect, the wearable user device, 102 can directly interact with the cloud and/or cloud servers and IoT devices. The data and/or information collected can be directly stored in the cloud server without taking any space on the user mobile and/or portable computing device. The mobile and/or portable computing device can directly interact with a server and receive information for feedback activation, trigger the feedback and deliver the feedback. Examples of the feedback include but not limited to auditory feedback, haptic feedback, tactile feedback, vibration feedback, or visual feedback from a primary wearable device, a secondary wearable device, a separate computing device (i.e. mobile), or IoT device (which may or may not be a computing device).

As used herein, the IoT devices can be a device that includes sensing and/or control functionality as well as a WiFi™ transceiver radio or interface, a Bluetooth™ transceiver radio or interface, a Zigbee™ transceiver radio or interface, an Ultra-Wideband (UWB) transceiver radio or interface, a WiFi-Direct transceiver radio or interface, a Bluetooth™ Low Energy (BLE) transceiver radio or interface, and/or any other wireless network transceiver radio or interface that allows the IoT device to communicate with a wide area network and with one or more other devices. In some embodiments, an IoT device does not include a cellular network transceiver radio or interface, and thus may not be configured to directly communicate with a cellular network. In some embodiments, an IoT device may include a cellular transceiver radio and may be configured to communicate with a cellular network using the cellular network transceiver radio.

A user may communicate with the computing devices using an access device that may include any human-to-machine interface with network connection capability that allows access to a network. For example, the access device may include a stand-alone interface (e.g., a cellular telephone, a smartphone, a home computer, a laptop computer, a tablet, a personal digital assistant (PDA), a computing device, a wearable device such as a smartwatch, a wall panel, a keypad, or the like), an interface that is built into an appliance or other device e.g., a television, a refrigerator, a security system, a game console, a browser, or the like), a speech or gesture interface (e.g., a Kinect™ sensor, a Wiimote™, or the like), an IoT device interface (e.g., an Internet-enabled devices such as a wall switch, a control interface, or other suitable interface), or the like. In some embodiments, the access device may include a cellular or other broadband network transceiver radio or interface and may be configured to communicate with a cellular or other broadband network using the cellular or broadband network transceiver radio. In some embodiments, the access device may not include a cellular network transceiver radio or interface.

In an embodiment, the users may be provided with an input/display screen which is configured to display information to the user about the current status of the system. The input/display screen may take input from an input apparatus, in the current example buttons. The input/display screen may also be configured as a touch screen or may accept input for determining vitals or bio-signals through touch or haptic based input system. The input buttons and/or screen are configured to allow a user to respond to input prompt from the system regarding needed user input.

The information which may be displayed on the screen to the user may be, for instance, the number of treatments provided, bio-signals values, vitals, the battery charge level, and volume level. The input/display screen may take information from a processor which may also be used as the waveform generator or a separate processor. The processor provides available information for display to the user allowing the user to initiate menu selections. The input/display screen may be a liquid crystal display to minimize power drain on the battery. The input/display screen and the input buttons may be illuminated to provide a user with the capability to operate the system in low light levels. Information can be obtained from a user through the use of the input/display screen.

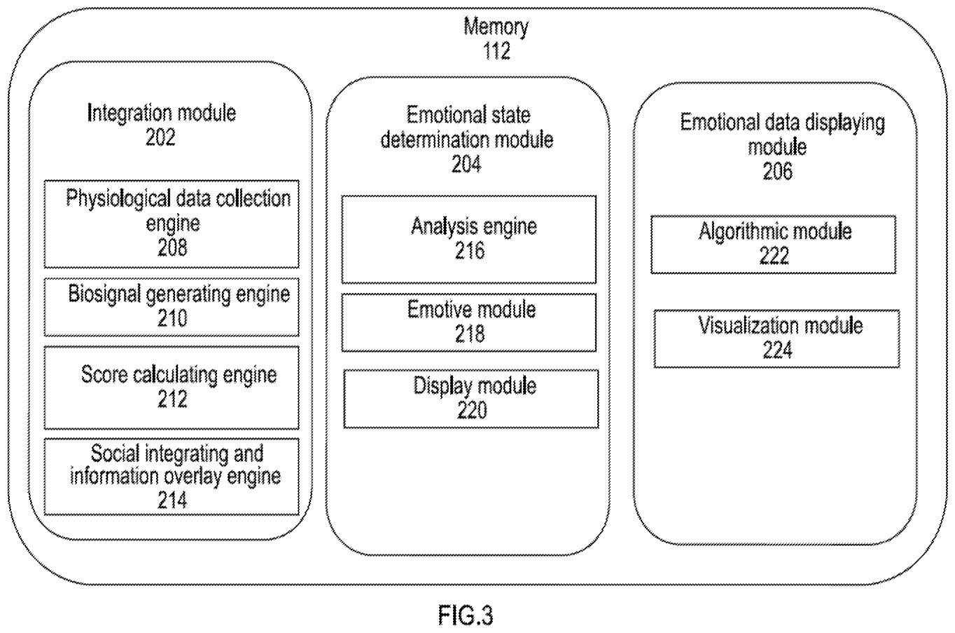

FIG. 3 illustrates a block diagram of the various modules within a memory 112 of a computing device, in accordance with another embodiment of the present invention. FIG. 3 is explained in conjunction with FIG. 1. The memory 110 includes an integration module 202, an emotional state determination module 204, and an emotional data displaying module 206.

The integration module 202 integrates the emotion data into the social network platform. The emotional state determination module 204 determines an emotional state of the user. The emotional data displaying module 206 analyzes and display emotional data of the users in real-time.

The integration module 202 includes a physiological data collection engine 208, a biosignal generating engine 210, a score calculating engine 212 and a social integrating and information overlay engine 214. The physiological data collection engine 208 collects physiological data of at least one physiological property of the user. The biosignal generating engine 210 processes the physiological data into at least one biosignal. The score calculating engine 212 monitors and measures the biosignal for determining at least one score pertaining to at least one of the emotions of the user, and stress of the user. The social integrating and information overlay engine 214 integrates the scores with at least one of a social media post associated with the social network platform, a textual conversation, and a multimedia conversation (audio, video) and overlays information pertaining to the emotion of the user, and stress of the user.

The emotional state determination module 204 includes an analysis module 216, an emotive module 218, and a display module 220. The analysis module 216 analyses the emotional state of the user on receiving the biorhythm data from the wearable user device. The emotive module 218 associates the analyzed emotional state of the user corresponding to at least one of one or more posts being shared by the user, one or more pieces of content being shared to the user, one or more reactions to the posts, and one or more responses to the posts over the social network platform. In an embodiment, the emotive module 218 facilitates the user to initiate a command to associate the emotional state of the user corresponding to the posts being shared by the user, and the content being shared to the user. The display module 220 displays a representation of the emotional state of the user in the social network platform. In an embodiment, the display module 220 displays the representation of the emotional state with respect to the post being shared by the user, and the content shared to the user on receiving a request command from the user.

The emotional data displaying module 206 includes an algorithmic module 222 and a visualization module 224. The algorithmic module 222 analyzes the biorhythm data and computes an emotional score of the user to generate one or more insights. The emotional score is indicative of the emotional state of the user during the interactions. The visualization module visually represents a plurality of emotional cycles for a specific time duration for the user. The visualization module 224 displays the insights and emotional scores of the users on the computing device associated with the users. In an embodiment, the visualization module 224 displays emotional data in a plurality of manners on at least one of a two dimensional (2D) graph, and a three dimensional (3D) graphs by using at least one of a plurality of alpha-numeric characters, a plurality of geometric shapes, a plurality of holograms, and a plurality of symbols which include colors or moving shapes.

The present specification further describes various use cases. In the first use case, the user is feeling fantastic in the present moment. However, in the next minute, the user came-across a post on social media or got a text from his friend or saw a random post on the news on the internet. Suddenly there is a change in emotions based on such new information. So, the change in the user's emotion needs to be determined and simultaneously shared with any secondary user.

In an embodiment, this change of emotions is dynamic and real-time. This change occurs because of the change in the state of another user. This change is determined by the biosensors connected to user s body wirelessly or by the wired medium. The change in biorhythm of the user determined the change and the change in emotions are conveyed to the communication channel to the second user if the user allows permission. In an embodiment, a threshold can be pre-determined, based on the threshold and breaching the threshold, the system and the method change the user communication to another channel to relax and/or calm the user. In an embodiment, the other user can also get the information based on the score about the user's emotions over the communication channel.

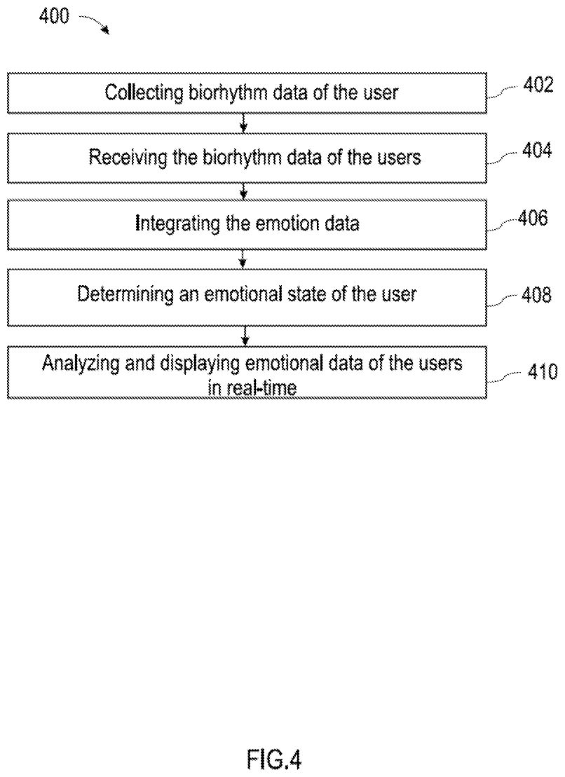

FIG. 4 illustrates a flowchart 400 of the method for integrating emotion data into a social network platform and share the emotion data over the social network platform connected through a communication network, in accordance with an alternative embodiment of the present invention. The method includes step 402 of collecting biorhythm data of the user through a wearable user device configured to be worn on the user's body, near the body, or placed in the user's body (implantable). The method includes the step 404 of receiving the biorhythm data of the users through a computing device communicatively connected with the wearable user device over the communication network. The method includes the step 406 of integrating the emotion data through an integration module. The method includes the step 408 of determining an emotional state of the user through an emotional state determination module. The method includes step 410 of analyzing and displaying emotional data of the users in real-time through an emotional data displaying module.

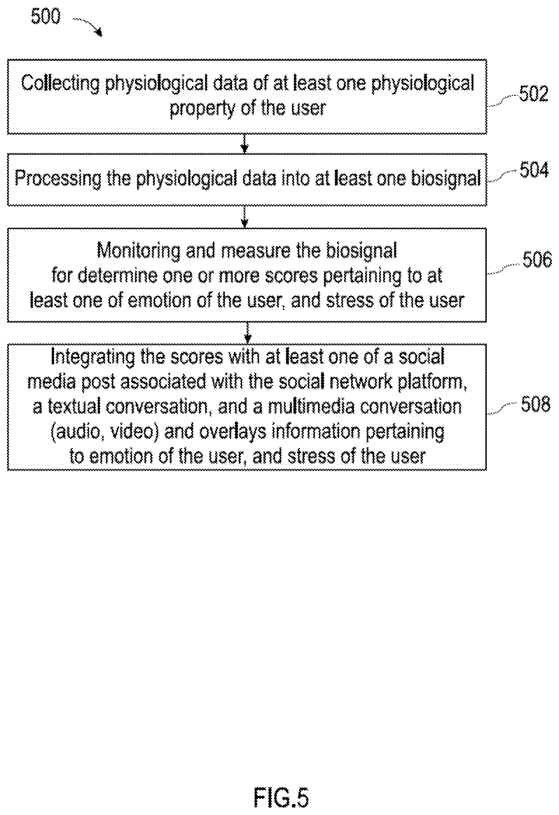

FIG. 5 illustrates a flowchart 500 of the plurality of steps performed by an integration module, in accordance with an alternative embodiment of the present invention. The integration module performs a plurality of steps that initiates with a step 502 of collecting physiological data of at least one physiological property of the user through a physiological data collection engine. The method includes the step 504 of processing the physiological data into at least one biosignal through a biosignal generating engine. The method includes the step 506 of monitoring and measures the biosignal for determining at least one score pertaining to at least one of the emotions of the user, and stress of the user through a score calculating engine. The method includes the step 508 of integrating the scores with at least one of a social media post associated with the social network platform, a textual conversation, and a multimedia conversation (audio, video) and overlays information pertaining to emotion of the user, and stress of the user through a social integrating and information overlay engine.

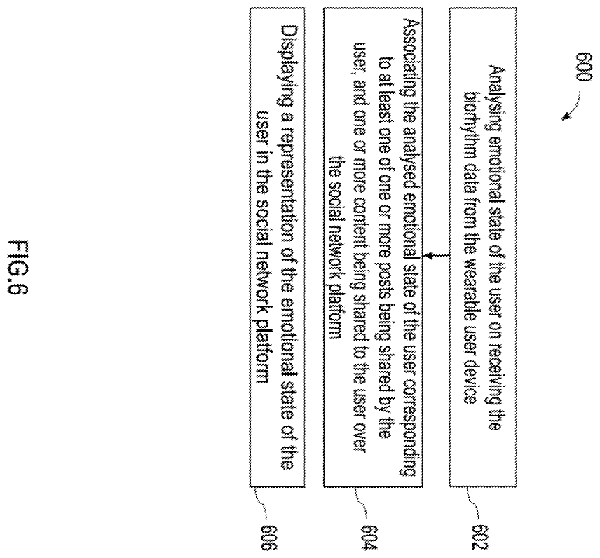

FIG. 6 illustrates a flowchart 600 of the plurality of steps performed by an emotional state determination module, in accordance with an alternative embodiment of the present invention. The emotional state determination module performs a plurality of steps that initiates with a step 602 of analyzing the emotional state of the user on receiving the biorhythm data from the wearable user device through an analysis module. The method includes the step 604 of one or more posts being shared by the user, one or more pieces of content being shared to the user, one or more reactions to the posts, and one or more responses to the posts over the social network platform through an emotive module. In an embodiment, the emotive module facilitates the user to initiate a command to associate the emotional state of the user corresponding to the posts being shared by the user, and the content being shared to the user. The method includes step 606 of displaying a representation of the emotional state of the user in the social network platform through a display module. In an embodiment, the display module displays the representation of the emotional state with respect to the post being shared by the user, and the content shared to the user on receiving a request command from the user.

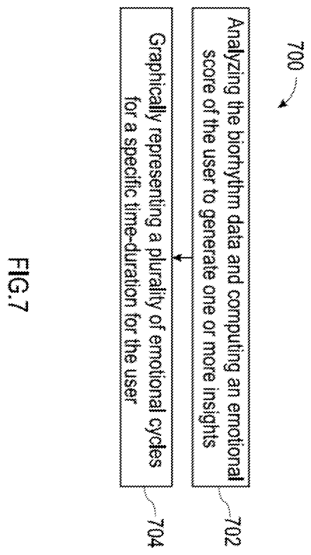

FIG. 7 illustrates a flowchart 700 of the plurality of steps performed by an emotional data displaying module, in accordance with an alternative embodiment of the present invention. The emotional data displaying module performs a plurality of steps that initiates with a step 702 of analyzing the biorhythm data and computing an emotional score of the user to generate one or more insights through an algorithmic module. The emotional score is indicative of the emotional state of the user during the interactions. The method includes the step 704 of visually representing a plurality of emotional cycles for a specific time duration for the user through a visualization module. The visualization module displays the insights and emotional scores of the users on the computing device associated with the users. In an embodiment, the visualization module displays emotional data in a plurality of manners on at least one of a two dimensional (2D) graph, and a three dimensional (3D) graphs by using at least one of a plurality of alpha-numeric characters, a plurality of geometric shapes, a plurality of holograms, and a plurality of symbols which include colors or moving shapes.

Thus the present invention provides a social network platform that allows the users to add more connections where upon an invitation is sent to other users for making connections. After acceptance from other users, the user could share data in various forms, but not limited to, photos, messages, attachments of various document file formats or image formats or video formats, audio clips, videos, animations/gifs. Based on the shared data or information, users could respond and share their emotions and feelings via the emoticons by clicking the corresponding buttons that represent, for example sad, happy, laugh, love and like. The system enables the user to request to associate the emotional state of the user with respect to at least one of the piece of content being shared by the user, and content being shared to the user, in the social network. The user could place the emoticons either adjacent to, superimposed on top of the piece of the content. The user could change the emoticons into semi-transparent or could use some other visual effect to respond for the piece of the content on the social networks.

While embodiments of the present invention have been illustrated and described, it will be clear that the invention is not limited to these embodiments only. Numerous modifications, changes, variations, substitutions, and equivalents will be apparent to those skilled in the art, without departing from the scope of the invention, as described in the claims.

1- A system to integrate emotion data into a social network platform and share the emotion data over the social network platform connected through a communication network, the system comprising:
a wearable user device to collect biorhythm data of the user; and
a computing device is communicatively connected with the wearable user device to receive the biorhythm data of the users over the communication network, wherein the computing device comprising:
a processor; and
a memory communicatively coupled to the processor, wherein the memory stores instructions executed by the processor, wherein the memory comprising:
an integration module to integrate the emotion data comprises:
a physiological data collection engine to collect physiological data of at least one physiological property of the user;
a biosignal generating engine to process the physiological data into at least one biosignal;
a score calculating engine to monitor and measure the biosignal for determining at least one score pertaining to at least one of the emotions of the user, and stress of the user; and
a social integrating and information overlay engine to integrate the scores with at least one of a social media post associated with the social network platform, a textual conversation, and a multimedia conversation and overlays information pertaining to the emotion of the user, and stress of the user;
an emotional state determination module to determine an emotional state of the user comprising:
an analysis module to analyze emotional state of the user on receiving the biorhythm data from the wearable user device;
emotive module to associate the analyzed emotional state of the user corresponding to at least one of one or more posts being shared by the user, one or more pieces of content being shared to the user, one or more reactions to the posts, and one or more responses to the posts over the social network platform; and
a display module to display a representation of the emotional state of the user in the social network platform; and
an emotional data displaying module to analyze and display emotional data of the users in real-time, wherein the emotional data displaying module comprising:
an algorithmic module to analyze the biorhythm data and compute an emotional score of the user to generate one or more insights, wherein the emotional score is indicative of the emotional state of the user during the interactions; and
a visualization module to visually represent a plurality of emotional cycles for a specific time-duration for the user, wherein the visualization module displays the insights and emotional scores of the users on the computing device associated with the users.

2- The system according to claim 1, wherein the emotive module facilitates the user to initiate a command to associate the emotional state of the user corresponding to the posts being shared by the user, and the content being shared to the user.

3- The system according to claim 1, wherein the display module displays the representation of the emotional state with respect to the post being shared by the user, and the content shared to the user on receiving a request command from the user.

4- The system according to claim 1, wherein the visualization module displays emotional data in a plurality of manners on at least one of a two dimensional (2D) graph, and a three dimensional (3D) graphs by using at least one of a plurality of alpha-numeric characters, a plurality of geometric shapes, a plurality of holograms, and a plurality of symbols.

5- A method for integrating emotion data into a social network platform and share the emotion data over the social network platform connected through a communication network, the method comprising steps of:
collecting biorhythm data of the user through a wearable user device;
receiving the biorhythm data of the users through a computing device communicatively connected with the wearable user device over the communication network;
integrating the emotion data through an integration module, wherein the integration module performs a plurality of steps comprising:
collecting physiological data of at least one physiological property of the user through a physiological data collection engine;
processing the physiological data into at least one biosignal through a biosignal generating engine;
monitoring and measuring the biosignal for determining at least one score pertaining to at least one of emotion of the user, and stress of the user through a score calculating engine; and
integrating the scores with at least one of a social media post associated with the social network platform, a textual conversation, and a multimedia conversation and overlays information pertaining to emotion of the user, and stress of the user through a social integrating and information overlay engine;
determining an emotional state of the user through an emotional state determination module, wherein the emotional state determination module performs a plurality of steps comprising:
analyzing the emotional state of the user on receiving the biorhythm data from the wearable user device through an analysis module;
associating the analyzed emotional state of the user corresponding to at least one of one or more posts being shared by the user, one or more pieces of content being shared to the user, one or more reactions to the posts, and one or more responses to the posts over the social network platform through an emotive module; and
displaying a representation of the emotional state of the user in the social network platform through a display module; and
analyzing and displaying emotional data of the users in real-time through an emotional data displaying module, wherein the emotional data displaying module performs a plurality of steps comprising:
analyzing the biorhythm data and computing an emotional score of the user to generate one or more insights through an algorithmic module, wherein the emotional score is indicative of the emotional state of the user during the interactions; and
visually representing a plurality of emotional cycles for a specific time-duration for the user through a visualization module, wherein the visualization module displays the insights and emotional scores of the users on the computing device associated with the users.

6- The method according to claim 5, wherein the emotive module facilitates the user to initiate a command to associate the emotional state of the user corresponding to the posts being shared by the user, and the content being shared to the user.

7- The method according to claim 5, wherein the display module displays the representation of the emotional state with respect to the post being shared by the user, and the content shared to the user on receiving a request command from the user.

8- The method according to claim 5, wherein the visualization module displays emotional data in a plurality of manners on at least one of a two dimensional (2D) graph, and a three dimensional (3D) graphs by using at least one of a plurality of alpha-numeric characters, a plurality of geometric shapes, a plurality of holograms, and a plurality of symbols.
